(19) United States
(12) Patent Application Publication
(10) Pub. No.: US 2002/0187506 A1
(43) Pub. Date: Dec. 12, 2002

(54) Syk localized at centrosome

(75) Inventor: Fatih M. Uckun, White Bear Lake, MN (US)

Correspondence Address: MERCHANT & GOULD PC, P.O. BOX 2903, MINNEAPOLIS, MN, 55402-0903, US

(73) Assignee: Parker Hughes Institute, St. Paul, MN

(21) Appl. No.: 10/159,696

(22) Filed: May 29, 2002

Related U.S. Application Data
(63) Continuation of application No. PCT/US00/32823, filed on Nov. 30, 2000.
(60) Provisional application No. 60/168,169, filed on Nov. 30, 1999.
(60) Provisional application No. 60/168,279, filed on Dec. 1, 1999.

(51) Int. Cl. C12Q 1/68; A61K 39/395

(52) U.S. Cl. 435/6; 424/155.1

(57) ABSTRACT

The method of the invention includes use of Syk, Syk-conjugte, Syk-binding ligand, or Syk inhibitor as a marker for the analysis of a cell's centrosome, centrioles, or mitiotic spindle, for example, in the diagnosis of cellular proliferation disorders, or for the study of cell cycle. In addition, the method of the invention included use of Syk-binding ligand or Syk inhibitors to inhibit the function of centrosome, centrioles, or mitiotic spindle, for example in the inhibition of cell proliferation, particularly cancer.

### BACKGROUND OF THE INVENTION

The Spleen Tyrosine Kinase (Syk) family of tyrosine kinase inhibitors are essential for hematopoeitic cell development and for signaling through the T-cell and B-cell antigen receptors as well as IgG and IgE Fc receptors. It has now been discovered that Syk, a tubulin kinase, is specifically associated with centrosomes, and mitotic spindles of B and T lineage hematopoietic cells, co-localizing with specific tubulins and additional specific centrosomal proteins.

Centrosomes, centrioles, and mitotic spindle architecture is essential for proper cell maintainance, including proper alignment of genetic material during mitosis and meiosis. Cellular proliferation is inhibited by impaired function of the mitotic spindle. Proper transfer of genetic information to daughter cells during mitosis or meiosis is compromised with malfunction of the spindle or centrioles.

Agents that provide analytical tools to accurately, efficiently, and economically assess centrosome, centriole, and spindle architecture in cells are needed. In addition, agents for accurately, efficiently, and economically disrupting the function of centrosome, centriole, and spindle architecture, and thereby disrupting cellular proliferation, for example, of cancer cells, are needed. The present invention provides agents that address these specific needs.

### SUMMARY OF THE INVENTION

Spleen Tyrosine Kinase (Syk) has now been discovered to specifically associate with centrosome, centrioles, and mitotic/meiotic spindle. Accordingly, agents that specifically bind to Syk, such as anti-Syk antibodies, Syk inhibitors, and the like, provide detectable labels for accurate, efficient, and economical marking and analysis of centrosome, centriole, or spindle architecture and function, in addition, agents that bind Syk, where inhibitory to Syk activity, provide a specific, efficient method for inhibiting centrosome, centriole, or spindle function, including inhibiting proliferation of cells, particularly cancer cells, and most particularly, cancer cells of hematopoietic origin.

It has also been discovered that Syk, for example as a labeled fusion protein, when administered to cells, becomes distributed to the centrioles and mitotic spindle. Accordingly, Syk itself is an agent that alone, or as a fusion protein, is useful as a marker agent for use in the analysis of centrosome, centriole, and spindle.

### BRIEF DESCRIPTION OF THE DRAWINGS

FIG. 1 is a computerized photograph showing a Western blot analysis of Syk and the cytoskeletal proteins alpha-tubulin, beta-tubulin, gamma-tubulin, and actin in the B-cell lines AB, KI-2, and Nalm6, as well as in the breast cancer cell line, BT-20.

FIGS. 2A-2C are computerized photographs showing results of immunocytochemical analysis of the B-cell lines KL-2 (FIG. 2A) and AB (FIGS. 2B and 2C) using labeled anti-Syk antibody. Syk staining was visualized in two small foci adjacent to the nucleus during interphase, marked with arrowheads in FIGS. 2A and 2B. In metaphase, Syk staining was observed in two small foci, one on each side of the metaphase plate (FIG. 2B, arrows). At higher magnification (FIG. 2C), the two foci demonstrate the organization of centrioles and individual triplets were observed. Scale bar=10 microns (FIGS. 2A, 2B); 5 microns (FIG. 2C).

FIGS. 3A and 3B are computerized photographs showing immunocytochemical localization of Syk to centrioles (FIG. 3A) and colocalization with the highly conserved centrosomal protein gamma-tubulin (FIG. 3B). Scale bar=10 microns

FIGS. 4A-4F are computerized photographs showing fractionation of centrosomal proteins from the B-cell line, AB. Results of Western blot analysis show Syk (FIG. 4A) was in the same fractions as the specific centrosomal proteins gamma-tubulin (FIG. 4B) and cep-135 (FIG. C). These fractions were also enriched in alpha- and beta-tubulin (FIG. 4D). Analysis with anti-phosphotyrosine antibody demonstrated the presence of polypeptides phosphorylated on tyrosine residues, including Syk (FIG. 4E). Paired centrioles stained with anti-Syk antibody are shown in the confocal microscopic image of fraction 6 (FIG. 4F).

FIGS. 5A-5F are computerized photographs showing confocal microscopic images of breast cancer cells stained with anti-Syk antibody. Shown are the following breast cancer cells: BT-20 (FIGS. 5A-5B); MCF-7 (FIGS. 5C-D); MDA-MD-231 (FIGS. 5E-5F). Nuclear staining (blue in the original), microtubule staining (red in the original), and Syk staining (green in the original) are shown in FIGS. 5A, 5C, and 5E. For clarity, only Syk staining is shown in FIGS. 5B, 5D, and 5F. Scale bar=20 microns

FIGS. 5A-6D are computerized photographs showing confocal microscopic images of BT-20 cells stained for Syk (FIGS. 5A and 5C) and for gamma-tubulin (FIGS. 5B and 5D). Shown are cells in interphase (FIGS. 5A and 5B) and in mitosis (FIGS. 5C and 5D). Scale bar=20 microns

FIGS. 7A-7F are computerized photographs showing confocal microscopic images of BT-20 cells stained for Syk. Labeling is shown for cells during interphase (FIG. 7A), prophase (FIG. 7B), prometaphase (FIG. 7C), metaphase (FIG. 7D), anaphase (FIG. 7E), and telophase (FIG. 7F). Scale bar=20 microns.

FIGS. 8A-8D are computerized photographs. Shown are results of Western blot analysis of glioblastoma cells (U373) transfected with Syk-GFP fusion construct. A 99 kd protein is identified with both anti-GFP antibody (FIG. 8A) and anti-Syk antibody (FIG. 8B). Immunocytochemistry shows the fusion protein localizing to the centrioles and at the plasma membrane of interphase cells (FIG. 8C), and to the mitotic spindle poles and fibers of mitotic cells (FIG. 8D). Scale bar=20 microns.

### DETAILED DESCRIPTION OF THE INVENTION

Method of the Invention

The present invention derives from the discovery that the spleen tyrosine kinase (Syk) specifically localizes to the centrioles, centrosomes, and/or mitotic spindle in cells. The invention exploits this discovery, by providing analysis of Syk at the centrosomes as a marker, or an indication of nuclear and cellular integrity. The methods of the invention also provides for the association or correlation of Syk specific cellular localization pattern with diagnosis or detection of normal and abnormal cellular state and disease. Disruption of Syk, e.g., via an inhibitor or antibody, and consequently of cell cycle progression and cellular proliferation are also methods of the invention.

It has been discovered that Syk localizes to the “cell center”, the centrosome. A centrosome is an organelle positioned near the nucleus of an animal cell that is the primary microtubuleorganizing center. The centrosome contains a pair of centrioles, each centriole is comprised of nine sets of triplet microtubules. The centrosome divides during mitosis, each centriole forming one of the spindle poles.

Localization of Syk in cells can be accomplished by specifically coupling a detection moiety to Syk. This detection moiety can be introduced in a number of ways, for example by contacting the cells with Syk, a Syk conjugate, a Syk binding ligand, or a Syk inhibitor. The detection moiety can either be covalently attached to Syk or can be attached by other bonds, such as coordinative, hydrogen bonding, ionic, or van der Waals bonding.

Conjugates can be created, for example, by having the detection moiety attached by a covalent bond. The detection moiety may be a polypeptide that is fused to the Syk protein, creating a Syk fusion protein. These fusion proteins may be Syk fused to the Green Fluorescence Protein (GFP) or Luciferase for example. Syk fusion proteins can be expressed from a plasmid transfected into the cell or can be expressed from a transgene present in the genome. Syk may also be covalently attached to a fluorphore, for example, a FITC or rhodamine molecule.

In another embodiment, the detection moiety can also be an antibody directed against Syk. The antibody can be a polyclonal or a monoclonal antibody and can have specificity against a desired region of the Syk protein. The antibody can be coupled to a fluorophore for detection or a different suitable detection moiety. The detection moiety can be a protein that interacts with Syk, such as a protein substrate. Since Syk is a kinase the detection moiety can be a protein or a peptide that interacts with Syk and is phosphorylated by Syk. This protein or peptide can be coupled to a fluorophore for detection or a different suitable detection moiety. The detection moiety can also be a small molecule that binds to Syk, such as an inhibitor or a cofactor. Preferably, the Syk inhibitor or cofactor is specific for Syk. This inhibitor or cofactor can be coupled to a fluorophore for detection or a different suitable detection moiety.

The function of Syk at the centrosome, centriole, and mitotic spindle can also be disrupted by contacting the cell with an inhibitor of Syk or a Syk binding partner that disrupts the function of Syk. This may be accomplished contacting cells with an inhibitor of Syk, for example piceatannol (CalBiochem), or an anti-Syc antibody. Syk function can also be disrupted by expressing, or over expressing, a Syk binding partner in the cell.

Detection of the location of Syk can be accomplished by microscopy techniques, for example, by confocal microscopy, by indirect immunofluorescence microscopy, or by electron microscopy using immunogold.

Definitions

Syk: Spleen tyrosine kinase, active to phosphorylate protein, particularly tubulin

Syk binding ligand: A ligand that specifically binds to Syk and may be used as a marker for Syk localization or may interfere with Syk function, acting as an inhibitor of its activity.

Syk inhibitor: An agent, which may be protein, small molecule, and the like, capable of inhibiting the functional activity of Syk, particularly its kinase activity at tubulin. One example of a Syk inhibitor is an anti-Syk antibody which may inhibit Syk activity.

Anti-Syk antibody: anti-Syk antibodies are generally known, and are commercially available, for example, Catalog No. sc-573, purified rabbit polyclonal IgG available from Santa Cruz Biotechnology (Santa Cruz, Calif.).

Other reagents useful in the method of the invention include the following:

| | |
|---|---|
| anti-alpha tubulin: | Cat # sc-8035 Santa Cruz Biotechnology (Santa Cruz, CA), |
| | purified mouse monoclonal IgM; clone TU-02 |
| anti-beta-tubulin | Cat # sc-7395 Santa Cruz Biotechnology (Santa Cruz. CA),, |
| | purified goat polyclonal IgG; |
| anti-gamma-tubulin | Cat # sc-7396 Santa Cruz Biotechnology (Santa Cruz, CA), |
| | purified goat polyclonal IgG; |
| anti-actin | Cat # A-4700 Sigma (St. Louis, MO), purified mouse monoclonal |
| | IgG2a. clone AC-40 |
| anti-phosphotyrosine: | Cat # 05-321 Upstate Biotechnonogy (Lake Placid, NY), purified |
| | mouse monoclonal IgG2bkappa; clone 4G10 |
| nucleus stain (blue) | DAPI (4′,6-diamidino-2-phenylindole, dihydrochloride); Cat # D- |
| | 1306, Molecular Probes (Eugene, OR) |
| microtubule stain (red) | Donkey Anti-goat Cy3-conjugate; Jackson |
| | Immunoresearch Cat # 705-165-147 |
| Syk stain (green) | Donkey Anti-Rabbit IgG Cy2 conjugate Jacksom |
| | Immunoresearch Cat # 711-225-152 |

### EXAMPLES

The Invention may be better understood with reference to the following examples:

### Example 1

### Analysis of Syk, Tubulins, and Actin in B-cell Lines and Breast Cancer Cells

The presence of Syk protein in B-cell lines AB, KL-2 and Nalm6 as well as in the breast cancer cell line BT-20, was analyzed by Western blot analysis. These cancer cell lines are available, for example, from the American Type Culture Collection and other public depositories.

Whole cell lysates were prepared using a 1% Nonidet-P40 lysis buffer, as described in Uckun et.al., 1996, Science 373:1096-1100. In brief, 30 microgram samples of whole cell lysates are loaded onto a 12% SDS-PAGE gel and the size-fractionated proteins are transferred onto a PVDF membrane (Millipore). The membrane is then blocked in 5% milk for at least one hour at room temperature, and then incubated with the appropriate antibody (dilution generally 1:1000) in PBS with 5% milk, overnight at 4° C. The blocked membrane is washed three times with PBST (150 mM NaCl, 16 mM Na₂HPO₄, NaH₂PO₄, 0.1% Tween, pH 7.3) at room temperature and incubated with a peroxidase-conjugated goat anti-rabbit IgG (1:2000 dilution) for two hours at room temperature. Immunoreactive proteins were detected by the enhanced chemiluminescence (ECL) system (Amersham), as described in Uckun et. al., Supra, and Sun et. al., 2000 PNAS, USA 96:680-685.

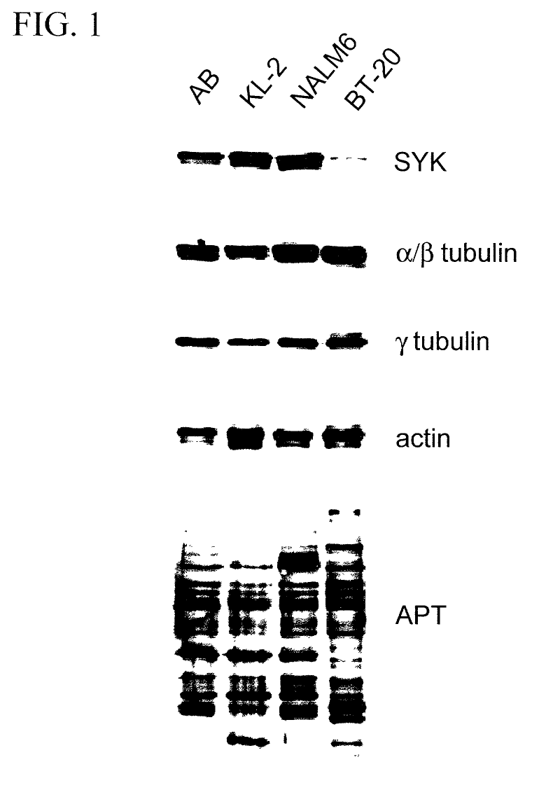

FIG. 1 shows the results of Western blot analysis of cancer cell lysates probed with anti-Syk antibody. Syk protein was found in each of the B-cell lines as well as in the breast cancer cell line, BT-20. The BT-20 cells showed proportionately less immunoreactive Syk protein than the B-cell lines, as evidenced by the comparative immunoreactivity of the cytoskeletal proteins alpha- and beta-tubulin and actin. All of the cancer cell lines demonstrated the presence of the centrosomal protein, gamma-tubulin.

### Example 2

### Immunocytochemical Localization of Sky in B-Cell Lines

The subcellular localization of Syk protein was examined by immunofluorescense and confocal laser scanning microscopy, as described in Uckun et. al. 1996, Supra, and Sun et. al. 1999, Supra. Briefly, cells were attached to poly-L-lysine coated glass coverslips by a 30 minute incubation at room temperature, washed twice with PBS, and fixed in ice cold (−20° C.) methanol for 15 minutes. In order to permeablize the cells and block the non-specific antibody binding sites, cells were treated with 0.1% Triton X-100 and 10% goat serum in PBS for 30 minutes.

To detect Syk protein, cells were incubated with anti-Syk antibody for one hour at room temperature. Cells were washed with PBS and incubated with a FITC-conjugated goat anti-rabbit IgG (Amersham)(1:40 final dilution) for one hour. Cells were then washed with PBS, counterstained with the DNA-specific nuclear dye toto-3 (Molecular Probes, Inc. 1:1000 dilution) for 10 minutes at room temperature, and washed again with PBS.

The coverslips were inverted, mounted onto slides in Vectashield (Vector Labs, Burlingame Calif.) to prevent photobleaching, and sealed with nail varnish as described in Sun et. al., 1999, Supra). Slides were examined using a a Bio-Rad MRC 1024 Laser Scanning Confocal Microscope mounted on a Nicon Eclipse E-800 upright microcope equipped for epifluorescence with high numerical aperature objectives, as described in Uckun et. al. 1996, Supra and Sun et. al., 1999, Supra. Optical sections were obtained and turned into stereomicrographs using Lasersharp software (BioRad). Representative digital images were processed using Adobe Photoshop software. Images were printed with a Fuji Pictography thermal transfer printer.

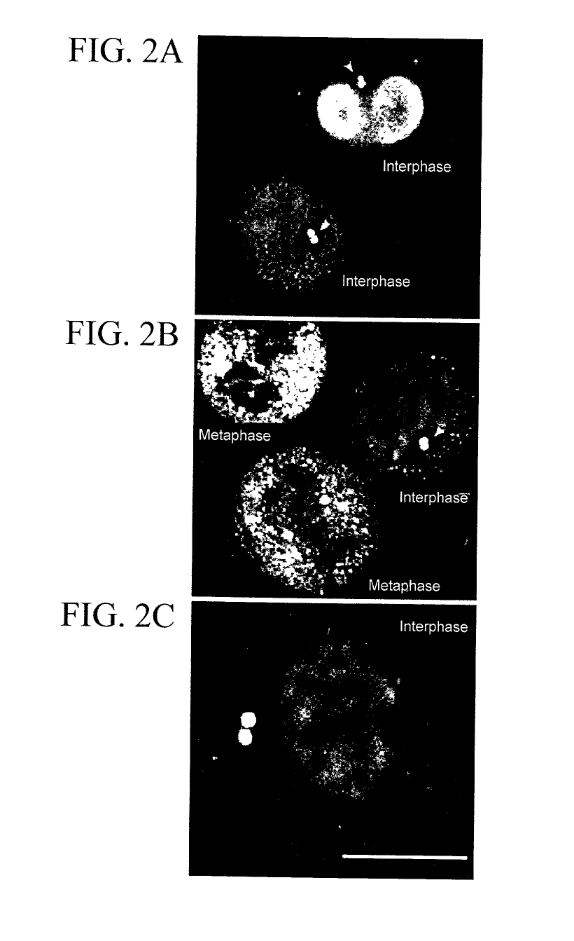

Results of the immunocytochemical analysis are show in FIGS. 2A-2C. In interphase cells, Syk staining (green in the original) was observed as two small foci adjacent to the nucleus (blue in the original) in both KL-2 cells (FIG. 2A, interphase, arrowheads, and AB cells (FIG. 2B, interphase arrowheads). In mitotic cells, identified by chromosomes aligned on the metaphase plate, one foci was observed on each side of the metaphase plate (FIG. 2B, arrows). At a higher magnification, the two foci have the organization of centrioles, and individual triplets are observed. This data verifies the utility of a Syk binding protein, an anti-Syk antibody, to view and analyze centrioles and spindle for architectural integrity.

### Example 3

### Co-localization of Syk and Gamma-tubulin in KL-2 Cells

Cells of the B-cell line KL2 were further analyzed for localization of Syk. The highly conserved centrosomal protein, gamma-tubulin was also analyzed. Cells were prepared and subjected to immunocytochemistry as described above for Example 2, using anti-Syk antibody and anti-gamma-tubulin antibody as probes.

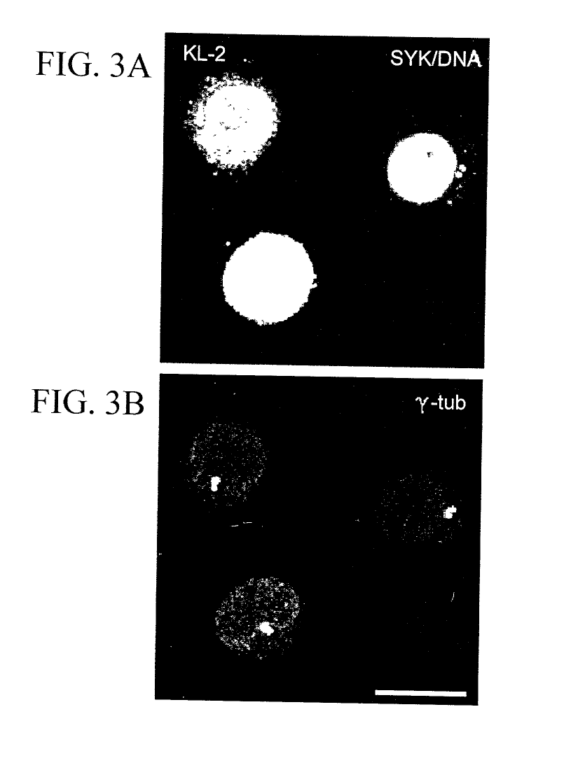

As shown in FIG. 3A, immunoreactive Syk (green in the original) was localized to centrioles, shown as two small foci. FIG. 3B demonstrates colocalization of Syk immunoreactivity with anti-gamma-tubulin antibody (red in the original).

### Example 4

### Fractionation of Isolated Centrosomal Proteins

Centrosomes may be isolated and analyzed generally following the methods described in Moudjou, M. & Bornens, M. (1994) in Cell Biology: A Laboratory Handbook, ed. Celis, J. E. (Academic, San Diego.), pp. 595-604. In brief, exponentially growing cells are incubated with culture medium containing 1 μg/ml cytochalasin D and 0.2 μM nocodazole for 1 hour at 37° C., to depolymerize actin and microtubule filaments. Cells are then trypsinized and lysed in a solution of 1 mM Hepes (pH 7.2), 0.5% Nonidet P-40, 0.5 mM MgCl2, 0.1% 2-mercaptoethanol with proteinase inhibitors (EDTA-free proteinase inhibitor cocktail, Boehringer Mannheim) and phosphatase inhibitors (50 mM sodium fluoride, 1 mM sodium orthovanadate). Swollen nuclei and chromatin aggregates are removed by centrifugation at 2,500×g for 10 minutes, and the supernatant is filtered through a 50-μm nylon mesh. Hepes is adjusted to 10 mM, DNase I (Boehringer Mannheim) added to 2 units/ml, and the mixture incubated for 30 minutes on ice. The lysate is next underlain with 60% sucrose solution (60% wt/wt sucrose in 10 mM Pipes, pH 7.2/0.1% Triton X-100/0.1% 2-mercaptoethanol).

Centrosomes are sedimented into the sucrose cushion by centrifugation at 10,000×g for 30 minutes. The crude centrosome preparation is purified further by discontinuous sucrose gradient centrifugation at 120,000×g for 1 hour. Usually 1-3×10⁷ cells are lysed in 5 ml of lysis buffer, and the cushion consists of 0.5 ml of 60% sucrose. After centrifugation, 1.5 ml from the bottom layer is resuspended and loaded onto a discontinuous gradient consisting of 500 μl of 70%, 300 μl of 50%, and 300 μl of 40% sucrose solutions. Fractions are collected from the bottom. e.g., 200 μl per fraction.

Each fraction is diluted in 1 ml of 10 mM Pipes buffer, pH 7.2. Centrosomes are recovered by centrifugation at 15,000 rpm for 10 minutes in a microfuge and denatured in SDS sample buffer (62.5 mM Tris, pH 6.8/10% glycerol/2% SDS/1.4% 2-mercaptoethanol/0.00% bromophenol blue).

Whole-cell lysates are prepared by sonicating cells briefly in a modified RIPA buffer (50 mM Tris, pH 8.0/150 mM NaCl/0.1% SDS/0.1% sodium deoxycholate/1% Nonidet P-40) with proteinase and phosphatase inhibitors, followed by centrifugation at 13,000 rpm for 10 minutes in a microfuge to remove cell debris, and denatured in SDS sample buffer. Proteins are separated by SDS/4-15% polyacrylamide gels and transferred to nitrocellulose membranes. Membranes are blocked in 100 mM Tris (pH 7.5) 150 mM NaCl, 0.1% Tween 20 (TBST) with 2% dry milk for 30 minutes at room temperature. Primary and secondary antibody hybridizations are carried out in TBST with 2% dry milk for 30-60 minutes; membranes are washed in TBST. Signals can be detected by using enhanced chemiluminescence (Amersham or Pierce).

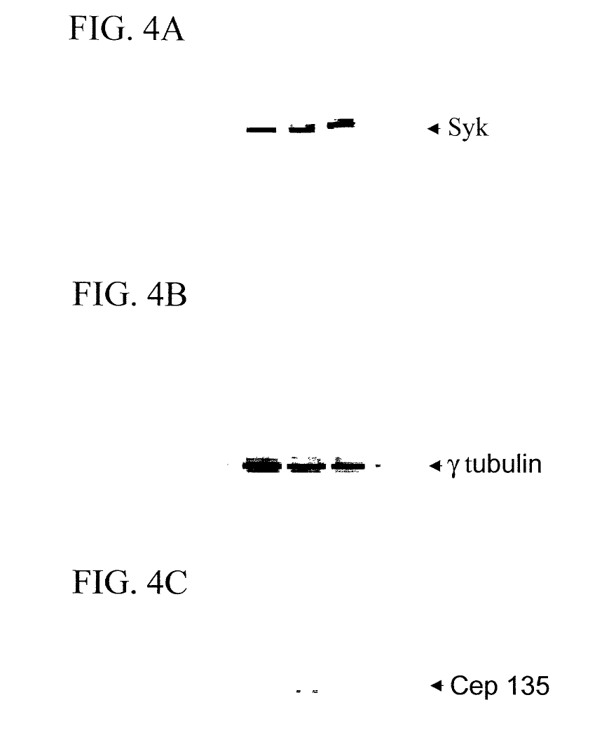

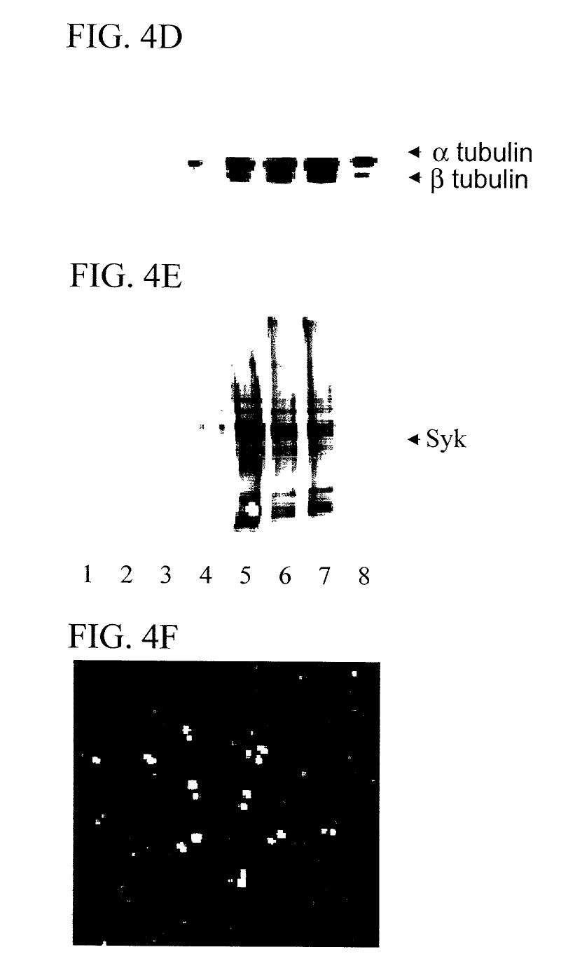

Centrosomal proteins were isolated from AB cells and fractionated for Western blot analysis. Cells were lysed and the centrosomal fractions were isolated and applied to SDS PAGE. The separated proteins were analyzed by Western blot for the presence of specific centrosomal proteins. The results are shown in FIGS. 4A-4E.

Syk (FIG. 4A) was found in the same fraction (fraction 6) of the preparation as two specific centrosomal proteins, gamma tubulin (FIG. 4B) and a novel centrosomal protein cep-135 (FIG. 4C). Fraction 6 was also enriched with the major tubulin isoforms, alpha and beta tubulin (FIG. 4D). Analysis with the anti-phosphotyrosine antibody showed the presence of polypeptides phosphorylated on the tyrosine, including Syk, in the preparation (FIG. 4E).

The enriched fraction 6 was then further analyzed by confocal microscopy using anti-Syk antibodies. As shown in FIG. 4F, paired centrioles were clearly labeled.

### Example 5

### Analysis of Sky in Breast Cancer Cell Lines

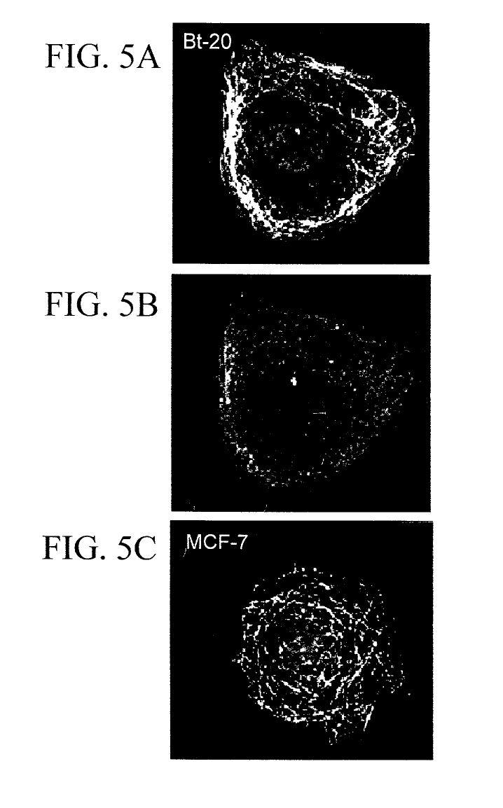

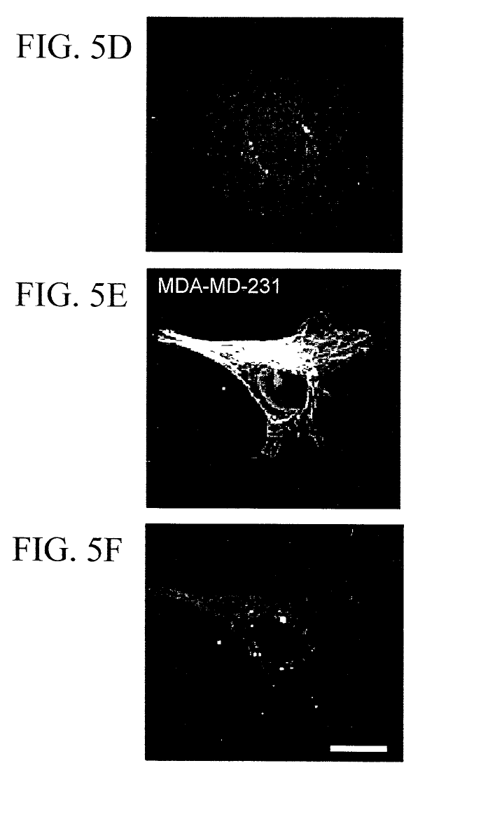

The presence of Syk in three separate breast cancer cell lines. BT-20, MCF-7, and MDA-MD-231 was examined by immunocytochemistry. As shown in FIGS. 5A-5F, Syk immunoreactive sites localized as two specific foci in the cells. FIGS. 5A, 5C, and 5E show additional nuclear (blue in the original) and tubulin (red in the original) staining. FIGS. 5B, 5D, and 5F show only Syk staining (green in the original), for clarity. This data demonstrates the localization of Syk in three different breast cancer cell lines, and further validates the use of Syk as a marker for analysis of centrioles and mitotic spindle.

### Example 6

### Colocalization of Sky and Gamma-tubulin in Breast Cancer Cells

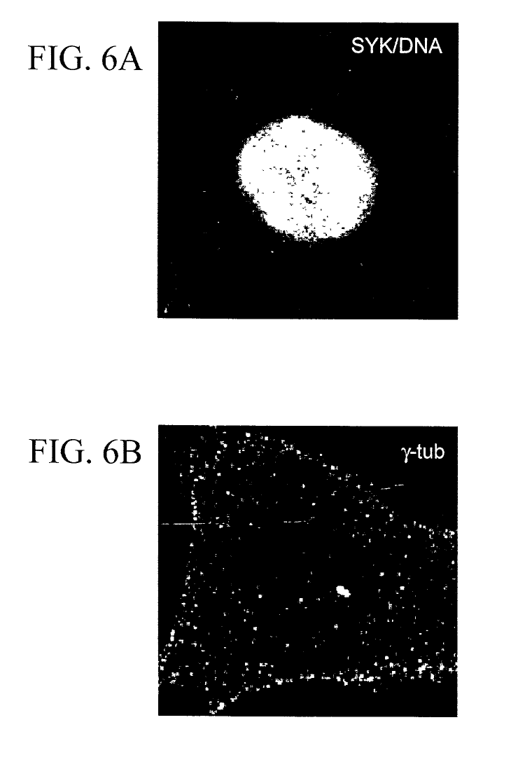

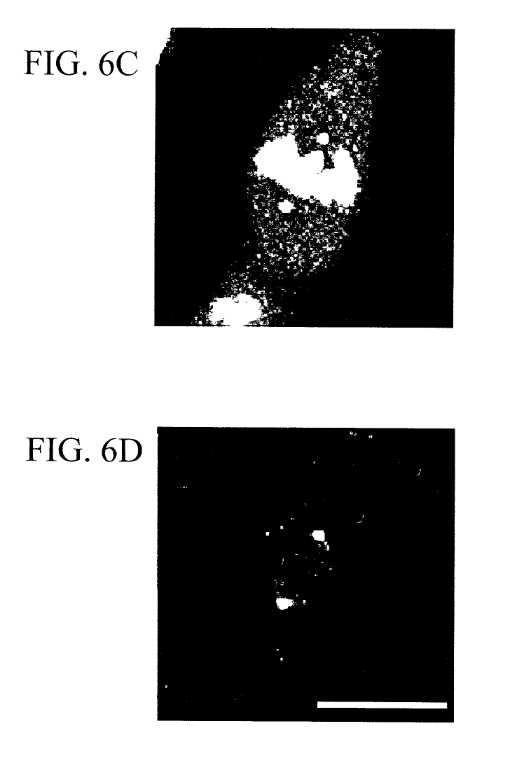

Breast cancer cells, BT-20, were analyzed for colocalization of Syk and gamma-tubulin by immunocytochemistry, viewing anaphase and metaphase stages of the cells. As shown in FIGS. 6A and 6B, both proteins were tightly associated with the centrioles during interphase. The stained centrioles migrated to opposing poles during metaphase, as shown in FIGS. 6C and 6D.

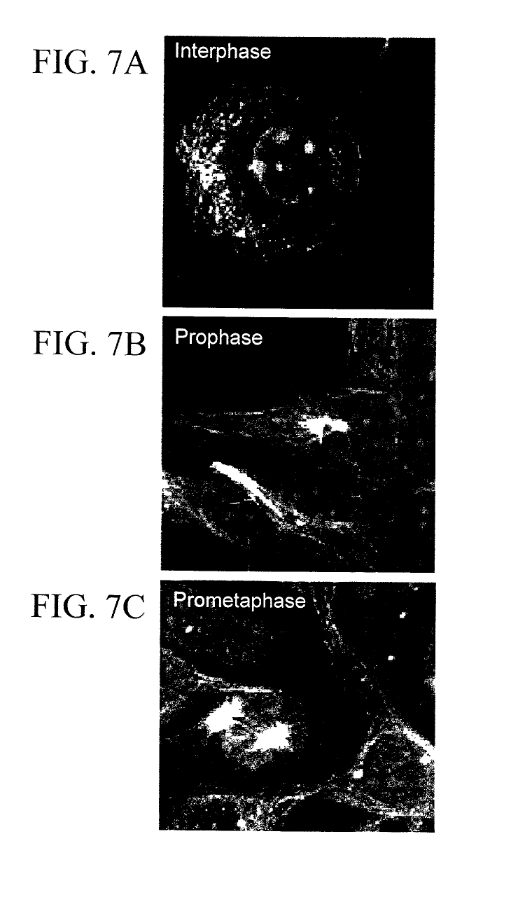

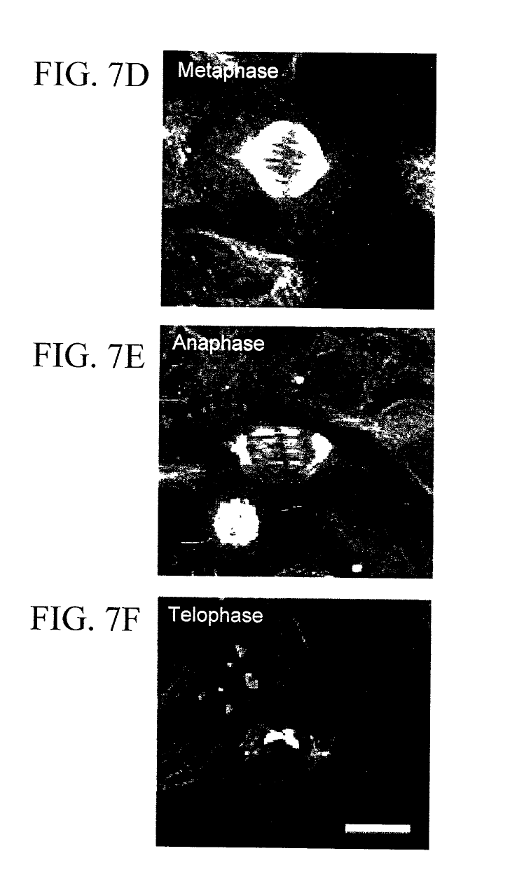

In a similar manner, cells were probed for the localization of Syk and gamma-tubulin proteins in cells through all stages of mitosis, from interphase through telophase. As shown in FIGS. 7A-7F, Syk staining was observed as bright dots adjacent to the nucleus in interphase BT-20 cells. When the duplicated chromosomes split during prophase, Syk labeling was observed in both centrosomes (FIG. 7B). This staining pattern persisted through prometaphase (FIG. 7C), evidenced by the breakdown of the nuclear envelope and chromosomal condensation. At metaphase, there is a single Syk foci at each spindle pole, and the spindle is clearly marked (FIG. 7D). This focal staining remained through anaphase (FIG. 7E) and telophase (FIG. 7F).

### Example 7

### Analysis of Glioblastoma Cells Transformed with a Syk-GFP Conjugate Protein

Cells from a glioblastoma cell line, U373, were administered Syk-GFP conjugate protein and analyzed for its localization by Western blot and by immunocytochemistry.

Green fluorescent protein can be commercially obtained, for example in the pEGFP-N1 vector available from Clontech, and conjugated to Syk protein. Glioblastoma cells were incubated in the presence of the conjugate protein, Syk-GFP. After incubation, the cells were washed, and prepared for immunologic analysis.

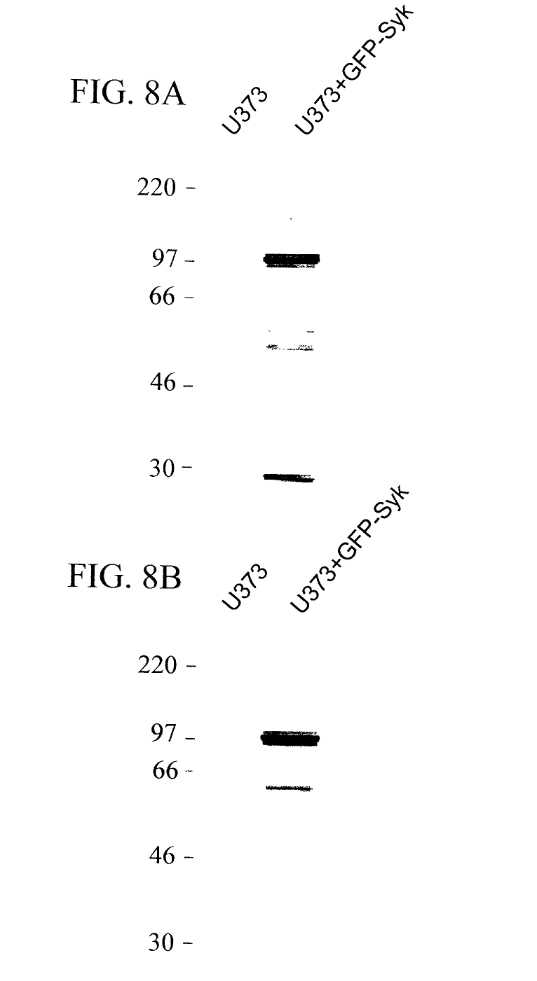

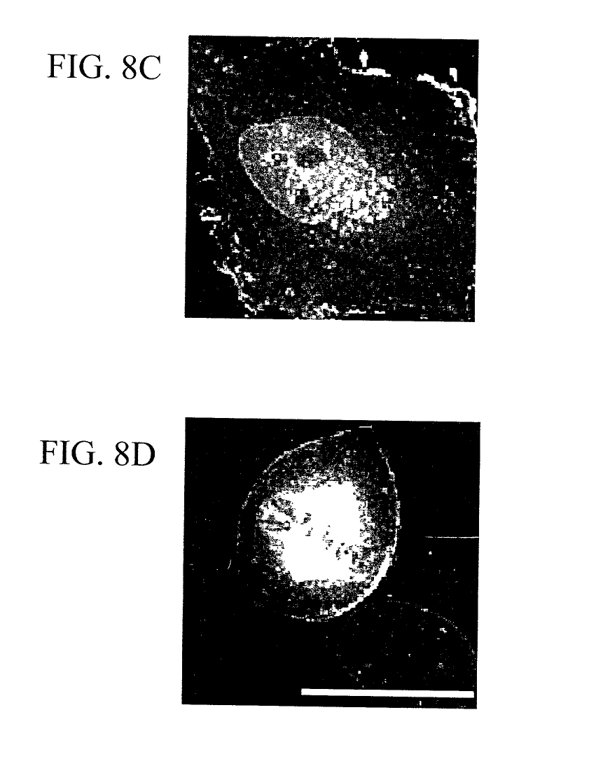

Western blot analysis was conducted as described above for Example 1. When probed with anti-Syk antibody (FIG. 8A) and anti-GFP antibody (FIG. 8B), a 99 kd protein was identified by each. By immunocytochemistry, the Syk-GFP conjugate localized to the centrioles of anaphase cells (FIG. 8C). In mitotic cells, the conjugate localized to the mitotic spindle poles and spindle fibers. Non-transfected cells did not react in Western blot, nor did they fluoresce in the immunocytochemistry assay. The data validates the use of Syk or Syk conjugate alone as a marker for centrioles and spindle integrity.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

We claim:

1. A method for analyzing spindle, centrosome, or centriole architecture or integrity in a cell, comprising:
contacting the cell with an effective analytical amount of Syk, Syk-conjugte, Syk-binding ligand, or Syk inhibitor; and
correlating cellular distribution of the Syk, Syk-conjugte, Syk-binding ligand, or Syk inhibitor with the architecture and integrity of the cell's spindle, centrosome, or centriole.

2. A method for labeling or marking a cell's centrosome, centrioles, or mitiotic spindle, comprising contacting the cell with an effective marking amount of Syk, Syk-conjugte, Syk-binding ligand, or Syk inhibitor.

3. A method for disrupting the function of a cell's centrosome, centrioles, or mitiotic spindle, comprising contacting the cell with an effective disruptive amount of a Syk-binding ligand or Syk inhibitor.

4. A method for inhibiting cellular proliferation comprising contacting cells with an effective inhibitory amount of a Syk-binding ligand or Syk inhibitor.

5. A method of modifying cell cycle progression comprising contacting a cell with a Syk binding ligand or Syk inhibitor.

6. A method of inhibiting mitosis or meiosis in a cell, comprising contacting the cell with an effective inhibitory amount of a Syk binding ligand or Syk.

7. The method of any of claims 1-6, wherein the Syk, Syk-conjugte, Syk-binding ligand, or Syk inhibitor comprises a detectable marker.

8. The method of claim 7, wherein the marker is a fluorescent marker.

9. The method of claim 1, wherein said Syk-conjugate is Syk-GFP.

10. The method of claim 6, wherein the cell is a cancer cell.
